(19) United States
(12) Patent Application Publication
(10) Pub. No.: US 2025/0117834 A1
(43) Pub. Date: Apr. 10, 2025

(54) Semi-Automated-Printed-Packing-Slip-Based-Online-Order to-Retail-Fulfillment-Management-System

(71) Applicant: Patrick Eugene Leavy, Oak Park, IL (US)

(72) Inventor: Patrick Eugene Leavy, Oak Park, IL (US)

(21) Appl. No.: 18/105,806

(22) Filed: Jul. 11, 2023

(51) Int. Cl. G06Q 30/0601; G16H 10/60; G16H 20/10

(52) U.S. Cl. CPC G06Q 30/0601; G16H 10/60; G16H 20/10

(57) ABSTRACT

A new Cannabis retail business method to more accurately and efficiently fulfill customer orders, comprising a standard Cannabis retail POS system, standard centralized retail Cannabis order management portal, and standard POS receipt printer.

As each retail Cannabis dispensary customer arrives, a designated greeter will print out (2) order packing slips, (1) for the customer, and (1) sent to print in the order fulfillment vault area (click on order in order management portal and select “print” option for greeter's POS printer and again for order fulfillment area printer). Unlike current Cannabis retail industry standards, which do not use printed packing slips for order picking & fulfillment, the printed packing slip allows for the order to be filled with more accuracy and speed by staff than from the current Cannabis industry standard of pulling the customer order from memory.

A new Cannabis retail business method to more accurately and efficiently fulfill customer orders, comprising a standard Cannabis retail POS system, standard centralized retail Cannabis order management portal, and standard POS receipt printer.

Add a “print order” option to the main screen option for each/every retail Cannabis order in the centralized Cannabis order management portal, if not already a present option, such that printed order packing slips may be produced by any staff member from any POS receipt printer by clicking on any order in the order management portal and selecting “print”.

As each retail Cannabis dispensary customer arrives, a designated greeter will print out (2) order packing slips, (1) for the customer, and (1) sent to print in the order fulfillment vault area (click on order in order management portal and select “print” option for greeter's POS printer and again for order fulfillment area printer).

The order fulfillment staff will then take the printed order packing slip to the product shelves to quickly and accurately pick the customer order for service to the customer at the checkout counter.

Unlike current Cannabis retail industry standards, which do not use printed packing slips for order picking & fulfillment, the printed packing slip allows for the order to be filled with more accuracy and speed by staff than from the current Cannabis industry standard of pulling the customer order from memory.

Additionally, sending a printed order packing slip to the order fulfillment area upon the customer's arrival, allows for the order to be picked before the customer arrives at the counter, allowing for decreased transaction and wait times, and facilitating higher Sales per hour.

whereby maximum daily retail cannabis customer volume and revenue may be increased staggeringly.

I. Integrating centralized corporate retail Cannabis order management systems to Cannabis retail POS systems by adding “print” option to command menu for any/every retail Cannabis order.

II. Enabling the immediate production of printed order packing slips from any POS through the order management portal (or multiples for multiple orders)
For improved customer check-in/management and Improved order picking accuracy by staff
Heightened speed of order fulfillment and service to customer by staff whereby maximum daily retail cannabis customer volume and revenue may be increased staggeringly.
Previous retail cannabis industry standards lacked printed packing slips and relied on handwritten order tickets or staff memory for retail order fulfillment.
